(19) United States
(12) Patent Application Publication
(10) Pub. No.: US 2022/0079427 A1
(43) Pub. Date: Mar. 17, 2022

(54) METHOD AND SYSTEM FOR THE STEREOENDOSCOPIC MEASUREMENT OF FLUORESCENCE, AND SOFTWARE PROGRAM PRODUCT

(71) Applicant: Olympus Winter & Ibe GmbH, Hamburg (DE)

(72) Inventor: Thorsten Juergens, Hamburg (DE)

(73) Assignee: Olympus Winter & Ibe GmbH, Hamburg (DE)

(21) Appl. No.: 17/477,843

(22) Filed: Sep. 17, 2021

(30) Foreign Application Priority Data
Sep. 17, 2020 (DE) 10 2020 124 220.4

(51) Int. Cl. A61B 1/04; A61B 1/00; A61B 1/313

(52) U.S. Cl. CPC A61B 1/043; A61B 1/3137; A61B 1/00193

(57) ABSTRACT

A method for the stereoendoscopic measurement of fluorescence in a tissue to which a fluorescent agent has been added, wherein a stereo-optical system of a stereo videoendoscope is directed toward a region of the tissue to be investigated, and the fluorescent agent is excited by means of an excitation light to emit florescent light that is detected by the stereo-optical system of the stereo videoendoscope in a pair of stereoscopic images, or a sequence of pairs of stereoscopic images, the method including: using an optical disparity of at least one pattern arising in the stereoscopic image pair or the stereoscopic image pairs, determining a distance of the tissue from the stereo-optical system by using a stereo base and a stereo angle of the stereo-optical system of the stereo videoendoscope, and normalizing the fluorescence signal with a normalization factor depending on the determined distance.

### CROSS-REFERENCE TO RELATED APPLICATION

The present application is based upon and claims the benefit of priority from DE 10 2020 124 220.4 filed on Sep. 17, 2020, the entire contents of which is incorporated herein by reference.

### BACKGROUND

### Field

The present disclosure relates generally to stereoendoscopes and more particularly to a method and a system for the stereoendoscopic measurement of fluorescence in a tissue to which a fluorescent agent has been added, wherein a stereo-optical system of a stereo videoendoscope is directed toward a region of the tissue to be investigated, and the fluorescent agent is excited by means of an excitation light to emit florescent light that is detected by the stereo-optical system of the stereo videoendoscope in a pair of stereoscopic images, or a sequence of pairs of stereoscopic images.

### Prior Art

Endoscopic measurements of fluorescence are performed in many medical areas of application. Typical of this is for example near-infrared fluorescence imaging (NIRF), which can be used to analyze and assess blood vessel perfusion, to confirm the anatomy of the hepatobiliary system, to find lymph nodes or to visualize the ureter following administration of an extrinsic contrast agent such as ICG (indocyanine green), CY5.5, ZW800 or ZW-1. Red dichromatic imaging (RDI) can be used to identify the source of arterial bleeding. Narrow band imaging (NBI) can help to differentiate between a benign hyperplasia and cancerous tissues or cancer precursors, for example between intestinal polyps of types NICE-1 and NICE-2, in order to decide whether or not a polyp needs to be resected.

In certain applications of fluorescence imaging such as in examining tissue perfusion, a quantification of the fluorescence signal may be in the interest of the user in order to determine the functional level at which wound healing or the sustained functionality of organs is improbable and a surgical procedure is therefore necessary.

The concepts associated therewith, both in terms of the need as well as thresholds and measuring approaches, have been discussed in the literature. Examples of this are inter glia:
Wada, T., et al. (2017), “ICG fluorescence imaging for quantitative evaluation of colonic perfusion in laparoscopic colorectal surgery,” Surgical endoscopy, 31(10), 4184-4193;
Kim, J C, et al. (2017), “Interpretative guidelines and possible indications for indocyanine green fluorescence imaging in robot-assisted sphincter-saving operations,” Diseases of the Colon & Rectum, 60(4), 376-384;
Hayami, S., et al. (2019), “Visualization and quantification of anastomotic perfusion in colorectal surgery using near-infrared fluorescence,” Techniques in coloproctology, 23(10), 973-980; and
Son, G. M., et al. (2019), “Quantitative analysis of colon perfusion pattern using indocyanine green (ICG) angiography in laparoscopic colorectal surgery,” Surgical endoscopy, 33(5), 1640-1649.

A problem in quantifying the fluorescence is the dependence on various factors, inter alia the dose of the circulating dye, which depends on the patient's blood volume and the injected amount of dye, tissue features, in particular the local anatomy of the patient, i.e., the thickness of the blood vessels and the thickness of other tissue layers that cover the blood vessels in the observed region, and the distance between the surface of the imaging device that emits the excitation light and also receives the emitted fluorescence light, and the observed object. The measured fluorescence signal decreases with increasing distance by the square of the distance.

To control the third factor, the measuring distance, several manufacturers of fluorescence imaging devices have developed approaches that however have their own disadvantages. These include inter alia the use of a fluorescence chart that can be used as a reference based on known object size and known fluorescence properties (dye concentration) in the chart. However, a reference chart is another disposable item that must be prepared for surgery, and introducing it into the surgical field can be associated with the risk of infection or the leaving of objects in the body of the patient.

An optical guidance can also be used, for example with two converging laser beams whose intersection marks the desired observation distance that should be used for reference measurements. However, an optical guidance using intersecting laser beams could restrict the versatility of the device since the observation distances cannot be freely selected. Moreover, additional components must be integrated into the camera system.

Another proposed option is the integration of a dedicated distance sensor for measuring the distance between the tissue and imaging device. However, a distance sensor also represents another component that drives up the costs of the device and could make the device larger or more unwieldy.

The distance problem is circumvented when a reference region (RR) and a region of interest (ROI) are defined, and a relative value for the ROI in comparison to the RR is measured. However, this requires that a reference region is available within the field of view (FOV). Even if a reference region is available, it could have a different distance to the imaging device than the ROI.

### SUMMARY

Accordingly, an object is to provide a robust and structurally simple solution for quantitative fluorescence signal measurement.

Such object can be achieved by a method for the stereoendoscopic measurement of fluorescence in a tissue to which a fluorescent agent has been added, wherein a stereo-optical system of a stereo videoendoscope is directed toward a region of the tissue to be investigated, and the fluorescent agent is excited by means of an excitation light to emit florescent light that is detected by the stereo-optical system of the stereo videoendoscope in a pair of stereoscopic images, or a sequence of pairs of stereoscopic images, wherein using an optical disparity of at least one pattern arising in the stereoscopic image pair or the stereoscopic image pairs, a distance of the tissue from the stereo-optical system is determined by using the stereo base and the stereo angle of the stereo-optical system of the stereo videoendoscope, and the fluorescence signal is normalized with a normalization factor depending on the determined distance.

A pattern is understood to be a structure that is contained in both images of the stereoscopic image pair, and is recognized as the same structure so that a determination of disparity is possible.

Many optical systems that are used for medical imaging are stereoscopic devices (such as WA50082A, LTF-S190-30, OrbEye). In a stereoscopic system, it is possible to determine observation distances based on the optical disparity. To accomplish this, one or a plurality of feature points can be detected in the image. By referencing these feature points in the left and right channels of the stereoscopic system, the disparity (offset of the two points in the imager coordinate system relative to each other) can be determined. Based on the known 3D parameters such as the stereo base and stereo angle, the distance of the feature points to the imaging device can be determined. The distance determined with a 3D system from the analysis of the disparity can be used to compensate for the fluorescence signal at different observation distances.

The normalization factor can be determined as a ratio of the square of the determined distance to the square of a standard distance. This takes into account the principle that the intensity of a signal decreases with the square of the distance from the source for geometric regions. Alternatively, instead of a direct functional dependence, calibration of the normalization factor can be made at the factory or while servicing. The normalization factor can be calculated using the determined distance, or taken from a lookup table.

In an embodiment of the method, the distance to the stereo-optical system can be determined for several points of the tissue, and a three-dimensional area is interpolated linearly or nonlinearly for the several points, or approximated with splines. Expressed otherwise, this means that the distance between the stereo-optical system and the observed field of vision can be measured, and a cloud of feature points, or respectively points of patterns, or respectively structures can be generated. A 3D surface can be interpolated from the cloud of feature points. The interpolation can be performed based on a linear interpolation, or based on a nonlinear interpolation method such as spline interpolation.

In an embodiment, the resulting three-dimensional area can be converted into a distance map that contains specific distances for specific pixels or pixel regions of one of the stereoscopic images or both stereoscopic images that accordingly correlates certain distances for individual pixels or pixel areas of the image or the images. Based on the distance, the variable normalization factor, or respectively amplification value, can be calculated or retrieved from a reference table that models the attenuation of the fluorescence signal depending on the observation distance. The fluorescence signal of each pixel, or respectively each pixel area can then be multiplied or divided by the distance-dependent normalization factor depending on the definition of the normalization factor. The result can be a homogenized fluorescence image that is compensated for different observation distances, including different observation distances within the field of view.

In another embodiment, the distance to the stereo-optical system can be determined for a predefined or adjustable, such as central, measuring field of the stereoscopic image, wherein the determined distance can be used to determine the normalization factor for the entire image. In so doing, the measuring field can have a linear extent of between 1% and 10% of the image height, and/or when there are several recognized patterns in the central region, an average of the distances determined for the different patterns can be used. The measuring field can be predefined, or adjusted or selected by the user. Several predefined sizes of the measuring region can exist such as for example 1% of the image height, 5% of the image height, 10% of the image height, etc. that the user can choose.

Based on this distance value, a variable normalization factor can be calculated or retrieved from a reference table that models the attenuation of the fluorescence signal depending on the observation distance. The fluorescence signal of the overall image can be then multiplied, or respectively divided by the distance-dependent normalization factor, depending on the definition of the normalization factor. The fluorescence signal can be substantially the signal strength of the fluorescent light in each pixel or pixel region measured as the difference from a base value without fluorescent light. This base value can be determined in a dark image for each pixel or each pixel region. The difference of the signal from the base value can be multiplied or divided by the normalization factor, depending on the definition of the normalization factor. The result can be a homogenized fluorescence image that is compensated for different observation distances.

In an alternative embodiment of the method, a distance of at least one predefined or selectable region of interest (ROI) of the tissue to the stereo-optical system, and additionally a reference distance of a predefined or selectable reference range (RR) of the tissue to the stereo-optical system can be determined, wherein the normalization factor for the fluorescence signal in the measuring region can be determined based on the difference or the ratio of the determined distance to the determined reference distance. The simultaneous measurement in an ROI and a reference region has the advantage in comparison to an absolute measurement without a reference region that other interferences such as individual features of the tissue or fluctuations in the dosage of the fluorescent dye are suppressed. Accordingly for example, a region of the tissue can be used as the reference region that is considered normal, whereas an altered region is characterized as an ROI.

The positions and sizes of the measuring fields for the ROI and RR can be predefined or set by a user. It is conceivable that there are several predefined sizes of the regions such as for example 1% of the image height, 5% of the image height, 10% of the image height, etc. that the user can choose. In the event that different feature points within the measuring region are recognized, an average can be formed.

Based on the distance values of the RR (reference region) and the ROI (region of interest), the difference in the observation distance between the ROI and RR can be calculated. Based on this difference, a variable normalization factor can be calculated or taken from a lookup table that models the attenuation of the fluorescence signal depending on the observation distance. This value can be used in calculating the relative fluorescence signal intensity between the ROI and the RR to eliminate differences based on different observation distances between the ROI and the RR. The normalization value in this case can be a function of both observation distances and can be a function of the ratio of the observation distances, such as the square of the ratio of the observation distances, a function of the difference of the observation distances, or a function approximating calibration measurements.

In another embodiment that can be combined with the aforementioned embodiments, an illumination intensity distribution can also be used in normalizing the fluorescence signals that is created during a calibration performed before the investigation or in a subsequent step. In this embodiment, the previously described embodiments of the method can be combined with a reference map that reflects the variable illumination strength over the field of view. The variable illumination intensity can be either measured after the production of the videoendoscope or during a calibration step at the beginning of the medical intervention and saved in a memory of the endoscopic video system. The brightness of a pixel, a pixel region or a measuring region can then be multiplied by a correction factor in order to take into account observed fluorescence signal differences that result from the differing illumination intensity.

In embodiments, corrected images or fluorescence values can be used in order to determine a maximum fluorescence, a maximum relative fluorescence, and/or a time until reaching a maximum fluorescence, a maximum relative fluorescence, or a fraction thereof.

Such object can also be achieved by a system for the stereoendoscopic measurement of fluorescence that comprises a stereo videoendoscope with a stereo-optical system and an excitation light source, as well as an evaluation unit configured to execute a previously described method.

Such object can also be achieved by a processor configured to perform the method described above and a non-transitory computer-readable storage medium storing instructions that cause the processor to perform the method.

The system, processor and the computer-readable storage medium can embody the same features, properties and advantages as the method.

### BRIEF DESCRIPTION OF THE DRAWINGS

Further features of the embodiments will become apparent from the description of the embodiments together with the claims and the attached drawings. Embodiments can fulfill individual features or a combination of several features.

The embodiments are described below, without restricting the general idea of the invention, based on exemplary embodiments in reference to the drawings, whereby we expressly refer to the drawings with regard to all details that are not explained in greater detail in the text. In the figures:

FIG. 1 illustrates a schematic representation of a system, and

FIG. 2 illustrates a schematic representation of the principle of measuring distance with a stereo videoendoscope.

In the drawings, the same or similar elements and/or parts are provided with the same reference numbers; a reintroduction will therefore be omitted.

### DETAILED DESCRIPTION

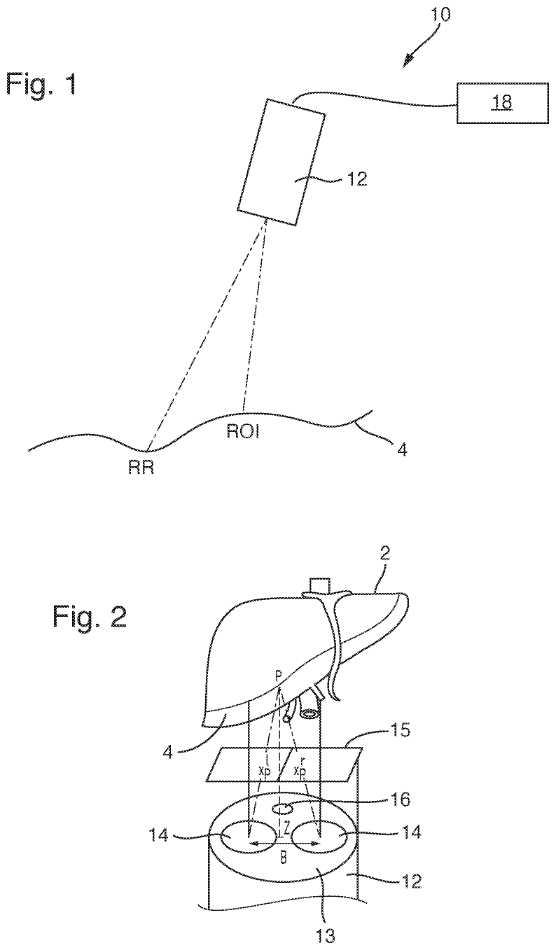

FIG. 1 shows a schematic representation of a system 10. The system 10 comprises a stereo videoendoscope 12 that is directed toward the tissue 4 of a patient and records stereoscopic image pairs with stereoscopically shifted images of the tissue 4 in the field of view of the stereo videoendoscope 12 and forwards them to an evaluation unit 18 (a processor comprising hardware or a combination of software and hardware). The tissue 4 is perfused, wherein a fluorescence dye is added to the blood that emits florescent light in the direction of the stereo videoendoscope 12 while being illuminated with an excitation light.

The stereoscopic image pairs thereby contain a qualitative image of the fluorescence activity of the imaged tissue 4. Since the intensity of the fluorescent light decreases with the square of the distance of the stereo videoendoscope 12 from the tissue 4, it is possible to eliminate the influence of the distance on the measured intensity of the fluorescent light from the tissue 4 by measuring the distance of the tissue 4 from the stereo videoendoscope 12 and using it to correct the fluorescence intensity.

FIG. 1 shows two regions of the tissue 4, namely a region of interest ROI and a reference region RR, between which a relative measurement is to be made. The aim of the measurement is to determine how much more intense or less intense the fluorescence is in the ROI compared to the RR. In comparison to absolute measurements, such a relative measurement has the advantage that other interferences such as individual features of the tissue 4 or fluctuations in the dose of the fluorescence dye are suppressed. Accordingly, for example, a region of the tissue 4 can be used as the reference region that is considered normal, whereas an altered region is characterized as an ROI.

In the example in FIG. 1, the distance of the stereo videoendoscope 12 from the reference region RR is 35% greater than the distance to the region of interest ROI. In order to render the intensity of the florescent light in the region of interest ROI comparable with that in the reference region RR, the intensity of the florescent light in the region of interest ROI is multiplied by the square of the ratio of the distances d_{RR} and d_{ROI}, i.e., 1.35⁻²=0.55.

FIG. 2 schematically portrays the principle of stereoscopically determining the distance. The distal tip 13 of the stereo-optical system 14 of the stereo videoendoscope 12 has two light entry windows, or respectively entry lenses for the left and the right stereo channel that have a basic distance B to each other. Furthermore, the distal tip 13 has a light exit opening 16 for an illumination light from which an excitation light exits to excite fluorescence in a fluorescence dye that was previously introduced into the tissue 4 of the organ 2.

The central beams from the two parallel optical systems face straight ahead and meet at two different points on the surface of the tissue 4 of an organ 2 that is being investigated. They can alternatively also have a known convergence angle relative to each other. A point P on the surface of the tissue 4 is perceived in the two channels at different angles relative to each other, and therefore passes through a projection plane 15 at different points x_{P}^{l}, x_{P}^{r}, or respectively arrives at different points on the image sensors (not shown) or image sensor regions for the left and the right channel. The two values x_{P}^{l}, x_{P}^{r} are offset relative to the center of the image by different amounts. This difference depends on the distance z of the point P from the stereo-optical system 14. When the basic distance B and the convergence angle between the two central beams of the left and right channel of the stereo-optical system 14 are known, the distance z can be calculated. The observation distance is determined in the evaluation unit 18 that also performs the image processing.

In this manner, the distances to one or more points on the surface of the tissue 4 of the organ 2 can be determined through stereoscopic disparity analysis. The normalization of the fluorescence signals can be carried out in various ways. Accordingly, in an instance in which the distance of the tissue 4 to the stereo-optical system 14 only varies insignificantly across the field of view of the stereo videoendoscope 12, a common normalization factor can be used for the entire image. When variations are larger, a model of the surface of the tissue 4 can be formed, and it can be determined for each pixel or different pixel regions which part of the surface is depicted therein, and a corresponding normalization factor can be chosen that corresponds to the distance to this part of the surface of the tissue 4.

Alternatively, a relative measurement can be performed with respect to a reference region RR as shown in FIG. 1.

In addition, an illumination intensity distribution can be taken into account in which both a known or previously determined illumination profile of the light source of the stereo videoendoscope 12 as well as the attenuation of the intensity of the illumination light depending on the distance of the tissue 4 from the distal tip 13 of the stereo videoendoscope 12 are also taken into account.

Due to the greater distance, regions of the surface of the tissue 4 that are more distant from the light exit opening 16 are excited with less illumination intensity to emit luminescent light than more closely lying regions. This attenuation of the illumination intensity follows the same functional dependence and progresses with the square of the inverse of the distance. Since both the illumination intensity as well as the intensity of the luminescent light absorbed by the videoendoscope decrease quadratically with increasing distance, the luminescence signal weakens with the inverse of the distance to the fourth power (d⁻⁴) in first approximation, assuming a homogeneous illumination profile.

In this regard, it can also be taken into account that outer regions of the field of view will be illuminated more weakly than central regions due to a possibly inhomogeneous illumination profile. Expressed simply, while strongly simplifying the geometric conditions, the attenuation can be approximated as a function which can be described with d⁻⁴ f(θ, φ) given a notation of the illumination profile f(θ, φ) in polar coordinates when the illumination light exits directly next to the entrance of the stereo-optical system 14 at the distal tip 13 of the video endoscope 12 as shown in FIG. 2.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMBERS

2 Organ
4 Tissue
10 System
12 Stereo videoendoscope
13 Distal tip
14 Stereo-optical system
15 Projection plane
16 Light exit opening
18 Evaluation unit

What is claimed is:

1. A method for the stereoendoscopic measurement of fluorescence in a tissue to which a fluorescent agent has been added, wherein a stereo-optical system of a stereo videoendoscope is directed toward a region of the tissue to be investigated, and the fluorescent agent is excited by means of an excitation light to emit florescent light that is detected by the stereo-optical system of the stereo videoendoscope in a pair of stereoscopic images, or a sequence of pairs of stereoscopic images, the method comprising:
using an optical disparity of at least one pattern arising in the stereoscopic image pair or the stereoscopic image pairs, determining a distance of the tissue from the stereo-optical system by using a stereo base and a stereo angle of the stereo-optical system of the stereo videoendoscope, and normalizing the fluorescence signal with a normalization factor depending on the determined distance.

2. The method according to claim 1, further comprising, calculating the normalization factor as a ratio of a square of the determined distance to a square of a standard distance.

3. The method according to claim 1, further comprising, calculating the normalization factor using the determined distance.

4. The method according to claim 1, further comprising retrieving the normalization factor a lookup table.

5. The method according to one of claim 1, wherein the determining of the distance of the tissue from the stereo-optical system comprises, determining, for several points of the tissue, and one of linearly or nonlinearly interpolating a three-dimensional area for the several points, or approximating the three-dimensional area with splines.

6. The method according to claim 5, further comprising, converting the three-dimensional area into a distance map that contains specific distances for specific pixels or pixel regions of at least one of the stereoscopic images.

7. The method according to claim 1, further comprising determining the distance to the stereo-optical system for a predefined or adjustable measuring field of the stereoscopic image, wherein the determined distance is used to determine the normalization factor for an entirety of the stereoscopic images.

8. The method according to claim 7, wherein the predefined or the adjustable measuring field of the stereoscopic image is a central measuring field of the stereoscopic image.

9. The method according to claim 8, wherein one or more of the measuring field has a linear extent of between 1% and 10% of the image height, and when there are several recognized patterns in a central region, an average of the distances determined for the different patterns is used.

10. The method according to claim 1, further comprising determining a distance of at least one predefined or selectable region of interest (ROI) of the tissue (4) to the stereo-optical system, and a reference distance of a predefined or selectable reference range (RR) of the tissue to the stereo-optical system, and determining the normalization factor for the fluorescence signal in the measuring region based on the difference or the ratio of the determined distance to the determined reference distance.

11. The method according to claim 1, further comprising using an illumination intensity distribution in normalizing the fluorescence signals created during a calibration.

12. The method according to claim 1, further comprising using corrected images or fluorescence values to determine one or more of a maximum fluorescence, a maximum relative fluorescence, a time until reaching a maximum fluorescence, a maximum relative fluorescence, and a fraction thereof.

13. A processor for stereoendoscopic measurement of fluorescence in a tissue to which a fluorescent agent has been added in a system comprising a stereo videoendoscope having a stereo-optical system configured to form a pair of stereoscopic images, or a sequence of pairs of stereoscopic images and an excitation light source configured to emit excitation light from the fluorescent agent;
the processor comprising hardware and being configured to use an optical disparity of at least one pattern arising in the stereoscopic image pair or the stereoscopic image pairs to:
determine a distance of the tissue from the stereo-optical system by using a stereo base and a stereo angle of the stereo-optical system of the stereo videoendoscope; and
normalize the fluorescence signal with a normalization factor depending on the determined distance.

14. A non-transitory computer-readable storage medium for stereoendoscopic measurement of fluorescence in a tissue to which a fluorescent agent has been added in a system comprising a stereo videoendoscope having a stereo-optical system configured to form a pair of stereoscopic images, or a sequence of pairs of stereoscopic images and an excitation light source configured to emit excitation light from the fluorescent agent;
the non-transitory computer-readable storage medium storing instructions that cause a processor to at least perform, using an optical disparity of at least one pattern arising in the stereoscopic image pair or the stereoscopic image pairs:
determining a distance of the tissue from the stereo-optical system by using a stereo base and a stereo angle of the stereo-optical system of the stereo videoendoscope; and
normalizing the fluorescence signal with a normalization factor depending on the determined distance.
